(19) United States
(12) Patent Application Publication
(10) Pub. No.: US 2009/0260624 A1
(43) Pub. Date: Oct. 22, 2009

(54) LIQUID EJECTION HEAD AND LIQUID EJECTION HEAD CARTRIDGE

(75) Inventors: HIROYUKI WADA, Machida-shi (JP); Masaru Sugita, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/422,490

(22) Filed: Apr. 13, 2009

(30) Foreign Application Priority Data
Apr. 16, 2008 (JP) 2008-106573

(51) Int. Cl. A61M 15/00

(52) U.S. Cl. 128/203.12

(57) ABSTRACT

A liquid ejection head to be used for an inhaler ejecting a liquid and making a user inhale the liquid includes: an ejection element having a liquid feeding opening, electrothermal transducer rows that are arrayed on both sides thereof respectively so as to sandwich the liquid feeding opening and are formed of a plurality of electrothermal transducers, and ejection nozzles for ejecting the liquid being provided on parts opposing to the electrothermal transducers respectively; and an external connecting terminal member for applying an electrical signal to the plurality of the electrothermal transducers, wherein, the external connecting terminal member is arranged so that a width direction in one end of its longitudinal direction matches with a direction of the electrothermal transducer rows and has a region for external connecting terminal on the other end of the longitudinal direction.

### BACKGROUND OF THE INVENTION

1. Field of the Invention

The present invention relates to a liquid ejection head for ejecting a liquid, which is used for an inhaler that is used for ejecting the liquid in a mist state and making the mist inhaled into the lungs.

2. Description of the Related Art

A medicine ejection apparatus (dispenser) is devised, which ejects a predetermined number of properly small droplets of a physiologically effective medicine into an air stream to be inhaled through a mouthpiece from a discharge orifice of an ejection head portion (ejection portion), and makes a user inhale the medicine. This medicine ejection apparatus imparts ejection energy to a liquid containing the medicine and ejects droplets by using a heater element or a piezoelectric element provided on the ejection head portion (ejection portion), similarly to a recording head which is used for an ink jet recording apparatus (see Patent Document 1 (Japanese Patent Application Laid-Open No. H08-511966)).

FIG. 9 illustrates a recording head which is disclosed in Patent Document 2 (US2007/0076060). This recording head mounts an ejection element 53 thereon which has one liquid feeding opening, electrothermal transducer rows which are arrayed respectively on both sides thereof and are formed of a plurality of electrothermal transducers, and has ejection nozzles 51 for ejecting a liquid, which are provided on parts opposing to the above described electrothermal transducers.

In addition, an external connecting terminal member 52 for applying an electrical signal to the plurality of the electrothermal transducers is arranged so that the longitudinal direction of the external connecting terminal member 52 matches with the direction of the above described electrothermal transducer rows.

FIGS. 10A and 10B illustrate a recording head which is disclosed in Patent Document 3 (Japanese Patent Application Laid-Open No. 2006-168233). This recording head mounts an ejection element H1100 thereon which has a liquid feeding opening, electrothermal transducer rows that are arrayed respectively in both sides thereof and are formed of a plurality of electrothermal transducers, and ejection nozzles for ejecting a liquid, which are provided on parts opposing to the electrothermal transducers respectively.

An external connecting terminal member H1300 for applying an electrical signal to the plurality of electrothermal transducers has a region for connecting terminals H1301 on one end in the longitudinal direction thereof, and has the above described electrothermal transducer rows arranged on the other end so that the direction of the rows matches with the longitudinal direction of the member (see Patent Document 4 (Japanese Patent Application Laid-Open No. 2007-320117)).

A liquid ejection head which is used in a conventional ink jet recording apparatus is structured while focusing on enhancing a resolution so as to obtain the printing of high quality, and extending the length of the ejection nozzle row so as to print information at a high speed.

However, a liquid ejection head to be used for an inhaler is required to have a smaller area of a face plane than the ink jet recording head, in order to make the inhaler into a portable size.

### SUMMARY OF THE INVENTION

The present invention is directed at providing a liquid ejection head which is used for an inhaler and has a downsized face plane so as to be a portable size.

In order to achieve the above described object, a liquid ejection head to be used for an inhaler which ejects a liquid and makes a user inhale the liquid includes: an ejection element which has a liquid feeding opening, electrothermal transducer rows that are arrayed on both sides thereof respectively so as to sandwich the liquid feeding opening and are formed of a plurality of electrothermal transducers, and ejection nozzles for ejecting the liquid, which are provided on parts opposing to the electrothermal transducers respectively; and an external connecting terminal member for applying an electrical signal to the plurality of the electrothermal transducers, wherein, the external connecting terminal member is arranged so that a width direction in one end of its longitudinal direction matches with a direction of the electrothermal transducer rows, and has a region for external connecting terminals on the other end of the longitudinal direction; and a plurality of the ejection elements having the same widths in the direction of the electrothermal transducer rows are arranged therein so that the widths are aligned.

The liquid ejection head according to the present invention has a structure as was described above, and accordingly shows an effect as will be described below.

In the liquid ejection head having a liquid feeding opening and a plurality of electrothermal transducers which are arrayed on both sides of the liquid feeding opening respectively, this liquid ejection head can decrease an area for electrical wiring on a face plane on which ejection nozzles are provided, and can decrease also an area of the face plane. As a result, the liquid ejection head can be downsized compared to a conventional ink jet recording head, and the obtained liquid ejection head can be suitable for an inhaler.

Other features and advantages of the present invention will become apparent from the following description of exemplary embodiments with reference to the attached drawings, in which like reference characters designate the same or similar parts throughout the figures thereof.

### BRIEF DESCRIPTION OF THE DRAWINGS

FIGS. 1A and 1B illustrate a liquid ejection head cartridge according to one embodiment of the present invention, in which FIG. 1A is a perspective view of an appearance thereof and FIG. 1B is a partial perspective view enlargingly illustrating an oval part of FIG. 1A.

FIG. 2 is an exploded perspective view of the liquid ejection head cartridge illustrated in FIGS. 1A and 1B.

FIG. 3 is a perspective view of the ejection element illustrated in FIG. 2, of which one part is ruptured.

FIGS. 4A and 4B are pattern views of an Si substrate illustrated in FIG. 3.

FIG. 5 is a view illustrating a method of attaching a liquid ejection head cartridge according to one embodiment of the present invention to an inhaler body.

FIGS. 6A and 6B are explanatory drawings showing an effect of a liquid ejection head according to one embodiment of the present invention, in which FIG. 6A is an explanatory drawing of the liquid ejection head illustrated in FIGS. 1A and 1B, which is viewed from a direction of an open arrow, and FIG. 6B is an explanatory drawing illustrating a conventional example.

FIGS. 7A and 7B illustrate a liquid ejection head cartridge according to another embodiment of the present invention, in which FIG. 7A is a perspective view of an appearance thereof and FIG. 7B is a perspective view enlargingly illustrating an oval part of FIG. 7A.

FIGS. 8A and 8B are explanatory drawings showing an effect of a liquid ejection head according to another embodiment of the present invention; FIG. 8A is an explanatory drawing of the liquid ejection head illustrated in FIGS. 7A and 7B, which is viewed from a direction of an open arrow, and FIG. 8B is an explanatory drawing illustrating a conventional example.

FIG. 9 is a perspective view of an appearance of an ink jet recording head according to a conventional example.

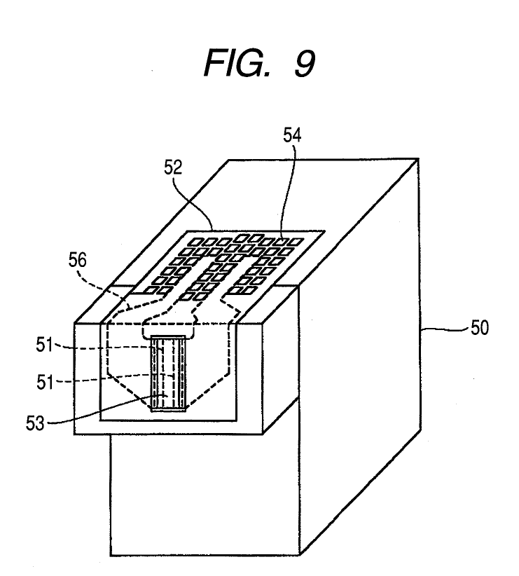

FIGS. 10A and 10B are partial perspective views of an ink jet recording head according to another conventional example.

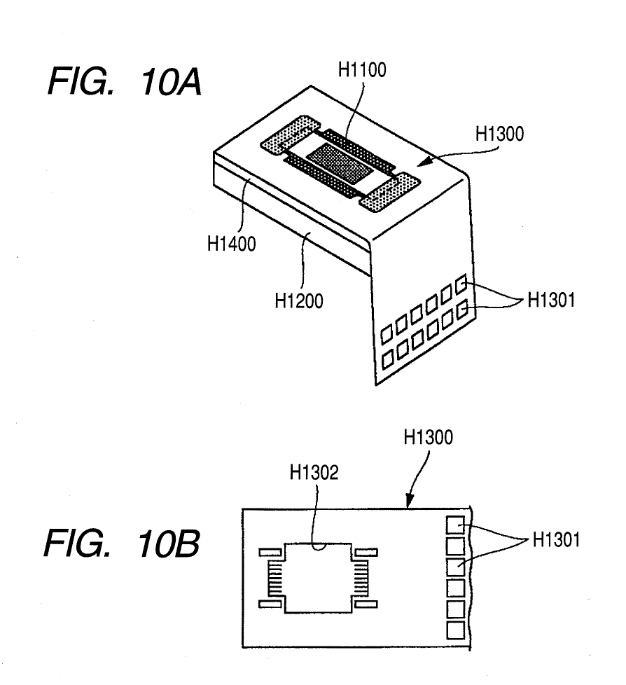

### DESCRIPTION OF THE EMBODIMENTS

Preferred embodiments of the present invention will now be described in detail in accordance with the accompanying drawings.

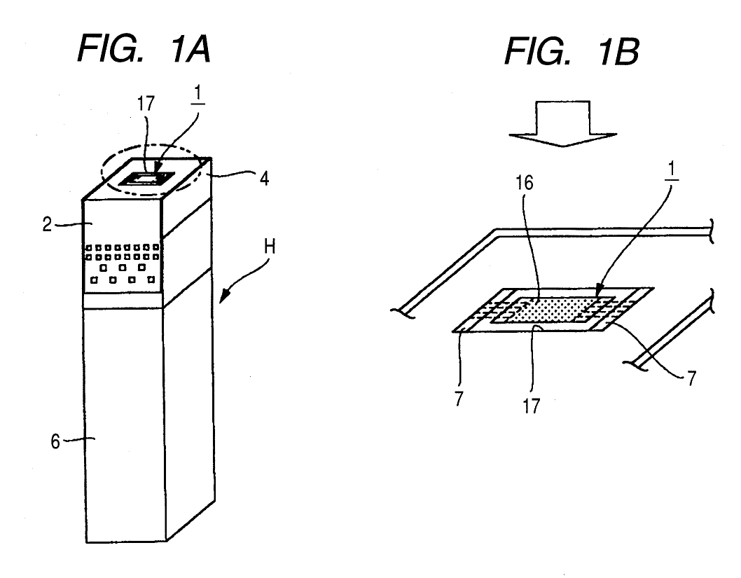

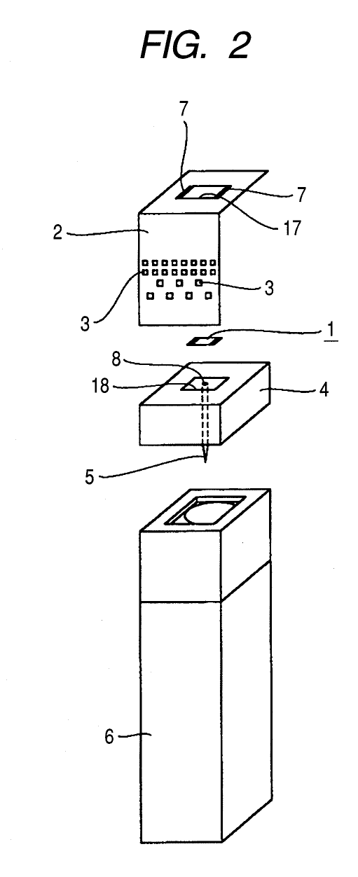

As is illustrated in FIGS. 1A, 1B and FIG. 2, a liquid ejection head is a side shooter type which ejects a liquid to a vertical direction from a face plane 16 by using an electrothermal transducer that generates thermal energy for causing the film boiling of the liquid according to an electrical signal.

An ejection element 1, an external connecting terminal member (tab film) 2, a chip holder 4 and a communication needle 5, which constitute a liquid ejection head, will now be described below sequentially.

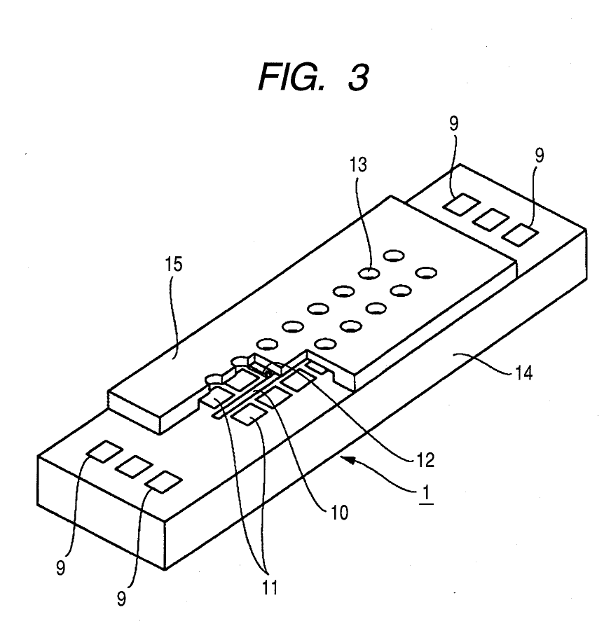

FIG. 3 is a perspective view of an ejection element 1 of which one part is ruptured. The ejection element 1 has a liquid feeding opening 10 which is a through port, for instance, having a long groove shape formed on an Si substrate 14 with a thickness of 0.5 to 1 mm as a liquid flow path, by a processing technique such as an anisotropic etching method using a crystal orientation of Si and a sand blasting method.

Electrothermal transducer rows formed of a plurality of electrothermal transducers 11 which are arrayed along the liquid feeding opening 10 with a distance from each other are arranged in each one row on both sides so as to sandwich the liquid feeding opening 10. These electrothermal transducers 11 and an electrical wiring such as AL for supplying an electric power to the electrothermal transducers 11 constitute one layer, and many layers thereof are formed with a film-forming technology. Furthermore, pads 9 for supplying an electric power to the electrical wiring are arranged on both sides so as to sandwich the electrothermal transducer rows formed of the plurality of the electrothermal transducers 11.

On the Si substrate 14, a liquid flow path wall 12 for forming liquid flow paths corresponding to the respective electrothermal transducers 11, and a nozzle plate 15 which has ejection nozzles 13 formed from a resin material or an inorganic material by a fine processing technique such as a photolithographic technology are arranged.

A liquid which has been supplied to the liquid flow path from the liquid feeding opening 10 is ejected by an air bubble which has been generated by thermal energy that has been imparted by the electrothermal transducer 11, from the ejection nozzles 13 provided on parts opposing to the electrothermal transducers 11.

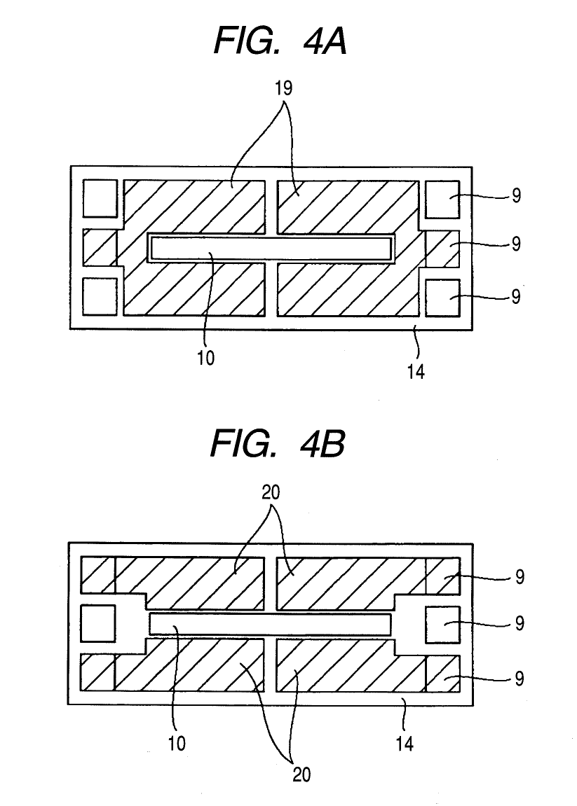

Next, an electrical wiring of AL or the like for supplying an electric power to an electrothermal transducer 11 will now be described. FIGS. 4A and 4B are views of a wiring pattern which is formed on an Si substrate 14. As is illustrated in FIG. 4A, a first layer wiring pattern 19 which is connected to any one of a plurality of pads 9 is connected to several electrothermal transducers 11 which are arrayed on both sides so as to sandwich a liquid feeding opening 10.

The several electrothermal transducers 11 which are connected to the first layer wiring pattern 19 are connected to a second layer wiring pattern 20 as is illustrated in FIG. 4B, and are further connected to a pad 9 to which the above described first layer wiring pattern 19 is not connected. Thus, the electric circuit is formed.

Next, a chip holder 4 will now be described with reference to FIG. 2.

The chip holder 4 is formed from a ceramic such as alumina or a metal material such as AL and SUS, and can employ a material having the same thermal conductivity or higher as the thermal conductivity of the material of an Si substrate 14 which constitutes an ejection element 1.

The chip holder 4 has a groove 18 which corresponds to a size of the ejection element 1 so as to mount the ejection element 1 thereon with high positional accuracy formed therein, and the ejection element 1 is bonded with the groove 18 by a first adhesive.

The first adhesive can have a low curing temperature, be cured in a short period of time, have a comparatively high hardness after having been cured, and have liquid resistance. An adhesive to be used therefor includes, for instance, a room temperature setting adhesive and a UV curing type adhesive which contain silicone as a main component.

The chip holder 4 further has a liquid supply hole 8 for supplying a liquid to the ejection element 1 formed on a part corresponding to a liquid feeding opening 10 of the ejection element 1. The liquid supply hole 8 penetrates from the surface of mounting the ejection element 1 thereon to the rear surface.

This liquid supply hole 8 makes a communication needle 5 penetrate therethrough, and the periphery of the penetrated portion is sealed by a second adhesive.

The second adhesive can have a low curing temperature, be cured in a short period of time, have a comparatively high hardness after having been cured, and have liquid resistance. An adhesive to be used therefor includes, for instance, a room temperature setting adhesive and a UV curing type adhesive which contain silicone as a main component.

Subsequently, an external connecting terminal member (tab film) 2 will now be described with reference to FIGS. 1A and 1B and FIG. 2. The external connecting terminal member 2 is a member for applying an electrical signal for ejecting a liquid, to an ejection element 1. The external connecting terminal member 2 has an opening portion 17 for incorporating the ejection element 1 therein, and an electrode terminal 7 corresponding to pads 9 of the ejection element 1.

The external connecting terminal member 2 also has an external connecting terminal region in which external connecting terminals (contact pin) 3 for receiving an electrical signal sent from a mainframe are arranged, provided on one end side in a longitudinal direction thereof. The electrode terminal 7 and the external connecting terminal 3 are connected through a wiring pattern of a continuous copper foil.

The electrode terminal 7 which connects an electrical wiring that extends from the external connecting terminal 3 through the external connecting terminal member 2 with the ejection element 1 is provided adjacent to the end in the direction of the electrothermal transducer row of the ejection element 1.

The external connecting terminal member 2 is electrically connected to the ejection element 1 through a connection unit. This connection unit can be a unit of electrically connecting the element, for instance, with an AL or Au wire by an ultrasonic thermocompression bonding method, or a unit which has an Au bump on a pad 9 of the ejection element 1, and electrically connects the Au bump to the electrode terminal 7 corresponding to the pad 9 by an ultrasonic thermocompression bonding method.

The pad 9 and the electrode terminal 7 which are electrically connected to each other are sealed with an unshown moisture-proof and insulation material so as to protect the connected portion.

The above described external connecting terminal member is arranged so that a width direction in one end of its longitudinal direction matches with the direction of the above described electrothermal transducer rows, and has a region for external connecting terminals on the other end of the longitudinal direction. Thereby, the external connecting terminal member 2 can be bent in a fold which is a virtual line parallel to the direction of the electrothermal transducer row that is arranged in the ejection element 1, and the rear surface thereof can be bonded to the side faces of a chip holder 4 and a reservoir 6 by using a third adhesive or a double-faced tape. Here, a “longitudinal direction” of the external connecting terminal member 2 in the present specification means a direction of a long side of the member in a state in which the external connecting terminal member 2 is not bent, and a “width direction” means a direction of a short side.

The third adhesive can have a low curing temperature, and can be cured in a short period of time. For instance, a hot-melt adhesive is used as the third adhesive.

Next, a liquid ejection head cartridge H will now be described with reference to FIG. 2. A reservoir 6 stores a medicine therein, and the liquid ejection head cartridge H is completed by inserting a communication needle 5 into the stored medicine and connecting the reservoir to the liquid ejection head.

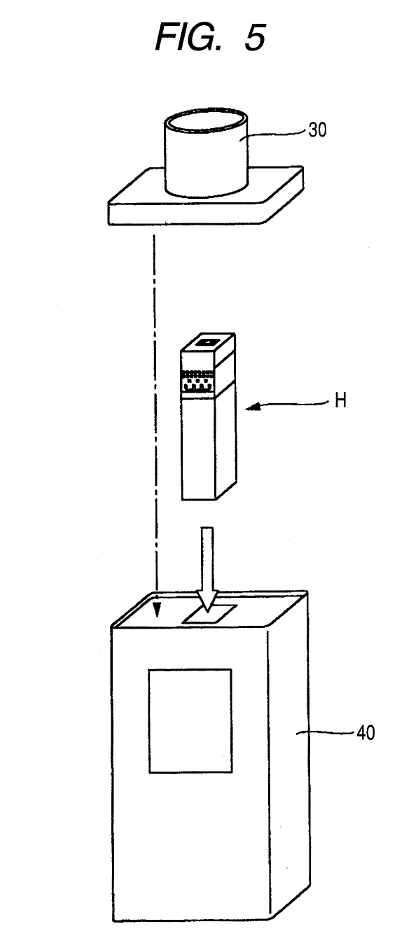

In other words, the liquid ejection head cartridge H has a liquid ejection head and the reservoir 6 which stores a liquid to be supplied to a liquid feeding opening 10, which are combined as a package. The liquid ejection head cartridge H is attached in a main body 40 of the inhaler as is illustrated in FIG. 5, and a mouthpiece 30 is mounted thereon. Thereby, a user can inhale the medicine.

In the present embodiment, the external connecting terminal member 2 is bent and the region for the external connecting terminals, in which an external connecting terminals 3 are provided, is provided in a direction different from a direction in which the liquid is ejected with respect to a face plane 16, so that the maximum contour of the liquid ejection head viewed from a direction in which the liquid is ejected can be reduced. Specifically, a plane including the region for the external connecting terminal forms an angle (right angle in the present embodiment) against the face plane.

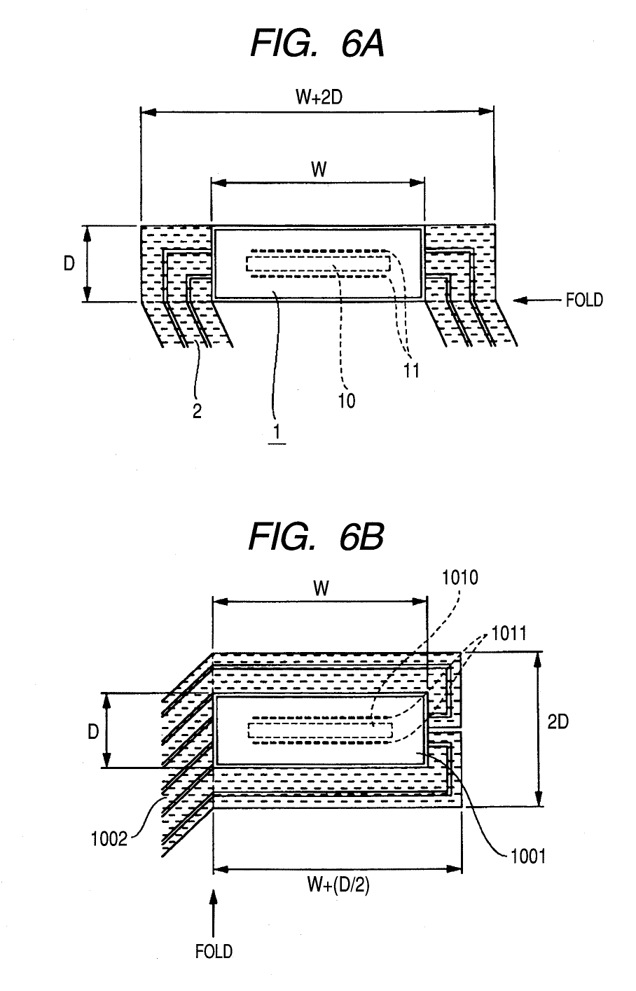

FIGS. 6A and 6B are explanatory drawings describing that an area of an electrical wiring on a face plane in one embodiment becomes smaller than that in a conventional example, in which FIG. 6A is an explanatory drawing of the liquid ejection head illustrated according to the present invention in FIG. 1, when viewed from a direction of an open arrow, and FIG. 6B is an explanatory drawing describing on the conventional example similarly to FIG. 6A.

In the present embodiment illustrated in FIG. 6A, when the width of an ejection element 1 is supposed to be W and the length of the ejection element 1 is supposed to be D, the area of the face becomes D×(W+2D), which is the total of electrical wiring areas in the ejection element 1 and an external connecting terminal member 2.

In contrast to this, in a conventional ejection head illustrated in FIG. 6B, when the width of an ejection element 1001 is supposed to be W and the length of the ejection element 1001 is supposed to be D, the area of the face becomes 2D×((W+(D/2)), which is the total of electrical wiring areas in the ejection element 1001 and an external connecting terminal member 1002. An electrothermal transducer 1011 is shown.

When the width W of the ejection element 1 is supposed to be 9 mm, and the length D of the ejection element 1 is supposed to be 3 mm, for instance, the area of the face becomes 45 mm² in the present embodiment, and the area of the face becomes 63 mm² in the conventional example, according to the above described area calculation formula. The area of the face in the present embodiment becomes approximately 29% smaller than that in the conventional example.

Furthermore, the difference between the areas of the faces becomes larger as a value of width W/length D of the ejection element 1 becomes larger.

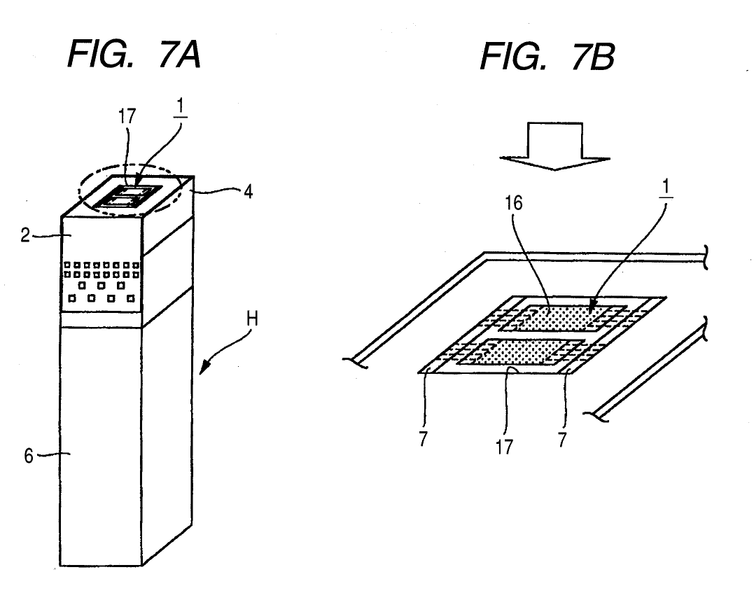

In the liquid ejection head according to the present invention, two or a plurality of ejection elements 1 are arranged as is illustrated in FIGS. 7A and 7B. In this case, a chip holder 4 may have liquid supply holes 8 for supplying a liquid to the respective ejection elements 1 formed on parts corresponding to liquid feeding openings 10 of the respective ejection elements 1.

Here, a plurality of ejection elements having equal widths in a direction of an electrothermal transducer row are arranged so that the widths are aligned. Thereby, an electrical wiring which extends to a contact pin 3 from the ejection element can be more compactly formed.

The plurality of the ejection elements can have the same size.

The plurality of the ejection elements can also be arranged adjacent to each other.

In addition, the same type of the medicine can be ejected from the plurality of the ejection elements. Thereby, an amount of the medicine to be ejected per unit time can be increased.

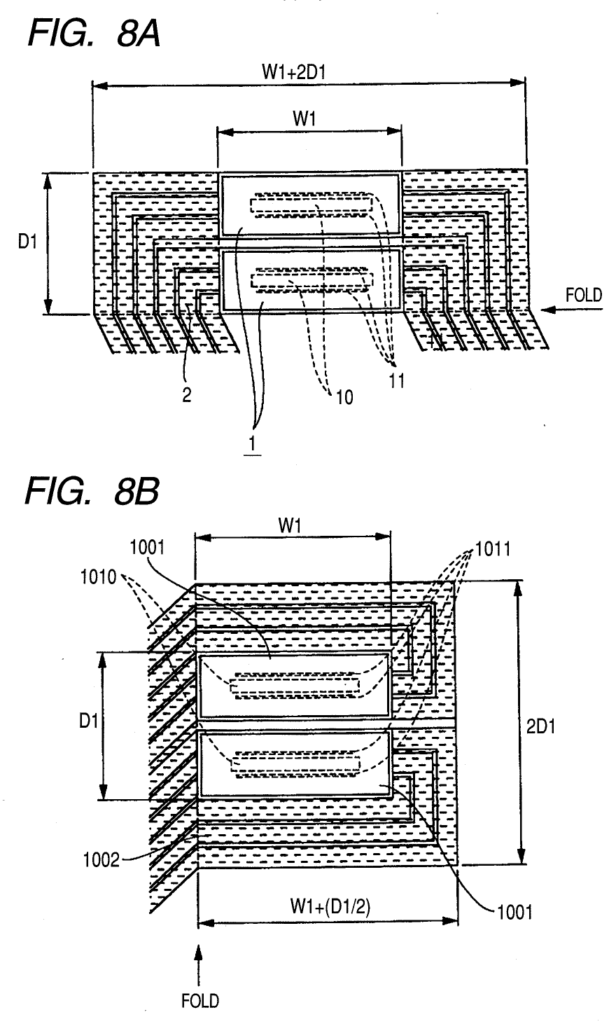

FIGS. 8A and 8B are explanatory drawings describing that an area of an electrical wiring on a face plane in the present invention becomes smaller than that in a conventional example, and an area of the face becomes small, in which FIG. 8A is an explanatory drawing of the liquid ejection head illustrated in FIGS. 7A and 7B, when viewed from a direction of an open arrow, and FIG. 8B is an explanatory drawing illustrating a conventional example.

In the present embodiment illustrated in FIG. 8A, when the width of a plurality of an ejection elements 1 is supposed to be W1 and the length of the plurality of the ejection elements 1 is supposed to be D1, the area of the face becomes D1×(W1+2D1), which is the total of electrical wiring areas in the ejection elements 1 and an external connecting terminal member 2.

In contrast to this, in a conventional ejection head illustrated in FIG. 8B, when the width of a plurality of an ejection elements 1001 is supposed to be W and the length of the plurality of the ejection elements 1001 is supposed to be D, the area of the face becomes 2D1×((W1+(D1/2)), which is the total of electrical wiring areas in the ejection elements 1001 and an external connecting terminal member 1002.

When the width W1 of the plurality of the ejection elements 1 is supposed to be 9 mm, and the length D1 of the plurality of the ejection elements 1 is supposed to be 7 mm, for instance, the area of the face becomes 161 mm² in the present embodiment, and the area of the face becomes 175 mm² in the conventional example, according to the above described area calculation formula. The area of the face in the present embodiment becomes smaller than that in the conventional example. Furthermore, the difference between the areas of the faces becomes larger as a value of width W1/length D1 of the plurality of the ejection elements 1 becomes larger.

A liquid ejection head and a liquid ejection head cartridge according to the present invention are not limited to the application for an inhaler of a medicine, but can be used in applications which need a hygienic liquid ejection, such as an inhaler for a flavoring agent and an inhaler of luxury goods like nicotine, for instance.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-106573, filed Apr. 16, 2008, which is hereby incorporated by reference herein in its entirety.

What is claimed is:

1. A liquid ejection head to be used for an inhaler which ejects a liquid and makes a user inhale the liquid comprising:
an ejection element which has a liquid feeding opening, electrothermal transducer rows that are arrayed on both sides thereof respectively so as to sandwich the liquid feeding opening and are formed of a plurality of electrothermal transducers, and ejection nozzles for ejecting the liquid which are provided on parts opposing to the electrothermal transducers respectively; and
an external connecting terminal member for applying an electrical signal to the plurality of the electrothermal transducers,
wherein,
the external connecting terminal member is arranged so that a width direction in one end of its longitudinal direction matches with a direction of the electrothermal transducer rows and has a region for external connecting terminal on the other end of the longitudinal direction, and
a plurality of the ejection elements having the same widths in the direction of the electrothermal transducer rows are arranged therein so that the widths are aligned.

2. The liquid ejection head according to claim 1, wherein the plurality of the ejection elements have the same size.

3. The liquid ejection head according to claim 1, wherein the region for the external connecting terminal is provided in a direction different from a direction in which the liquid is ejected.

4. The liquid ejection head according to claim 1, wherein the plurality of the ejection elements are provided adjacent to each other.

5. The liquid ejection head according to claim 1, wherein the plurality of the ejection elements eject the same type of the medicine.

6. The liquid ejection head according to claim 1, wherein an electrode terminal which connects an electrical wiring that extends from the external connecting terminal through the external connecting terminal member with the plurality of the ejection elements is provided adjacent to an end in a direction of the electrothermal transducer row of the ejection element.

7. A liquid ejection head cartridge comprising:
the liquid ejection head according to claim 1; and a reservoir that stores the liquid to be supplied to the liquid feeding opening, which are combined as a package.
